(19) United States
(12) Patent Application Publication
(10) Pub. No.: US 2025/0145562 A1
(43) Pub. Date: May 8, 2025

(54) VISIBLE LIGHT ABSORBERS FOR OPHTHALMIC DEVICES

(71) Applicant: Bausch + Lomb Ireland Limited, Dublin (IE)

(72) Inventors: Walter Laredo, Fort Worth, TX (US); Jeffrey Linhardt, San Francisco, CA (US)

(21) Appl. No.: 18/883,193

(22) Filed: Sep. 12, 2024

Related U.S. Application Data
(60) Provisional application No. 63/546,988, filed on Nov. 2, 2023.

(51) Int. Cl. C07C 245/08; A61F 2/16; C08F 220/30; G02C 7/10

(52) U.S. Cl. CPC C07C 245/08; A61F 2/16; C08F 220/306; G02C 7/104; A61F 2002/1699; C08F 2800/20

(57) ABSTRACT

A visible light absorbing azo compound having a structure of Formula I:

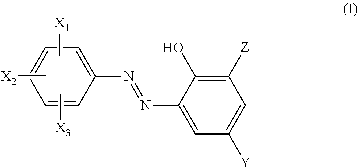

wherein X₁, X₂, X₃, Y and Z are as defined herein, and ophthalmic devices formed therefrom.

### PRIORITY CLAIM

The present application claims priority to U.S. Provisional Patent Application Ser. No. 63/546,988, entitled “Visible Light Absorbers for Ophthalmic Devices,” filed Nov. 2, 2023, the content of which is incorporated by reference herein in its entirety.

### BACKGROUND

Ultraviolet (UV) light absorbers are known components for polymeric materials used to make ophthalmic lenses and, in particular, intraocular lenses. The UV light absorbers are preferably polymerizable so as to be covalently bound to the polymeric network of the lens material instead of simply physically entrapped in the material, thereby preventing them from migrating, phase separating or leaching out of the lens material. Such stability is particularly important for implantable ophthalmic lenses where the leaching of the UV light absorber may present both toxicological issues and lead to the loss of UV light blocking activity in the implant. It has been shown that short wavelength visible light, both violet and blue, is damaging to cells both in vitro and in vivo.

### SUMMARY

In accordance with an illustrative embodiment, a visible light absorbing azo compound having a structure of Formula I:

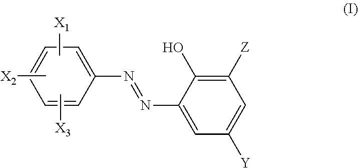

wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group.

In accordance with another illustrative embodiment, an ophthalmic device is a polymerization product of a monomeric mixture comprising:
(a) a visible light absorbing azo compound having a structure of Formula I:

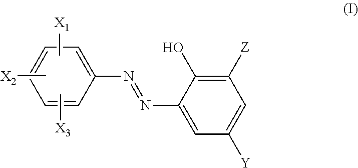

wherein X₁, X₂, X₃, Y and Z are as defined herein, and
(b) one or more ophthalmic device-forming monomers.

In accordance with yet another illustrative embodiment, a method for making an ophthalmic device comprises:
(a) providing a monomeric mixture comprising (i) a visible light absorbing azo compound having a structure of Formula I:

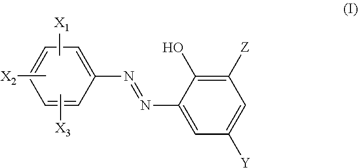

wherein X₁, X₂, X₃, Y and Z are as defined herein, and (ii) one or more ophthalmic device-forming monomers, and
(b) subjecting the monomeric mixture to polymerizing conditions to provide an ophthalmic device.

### BRIEF DESCRIPTION OF THE DRAWINGS

In combination with the accompanying drawing and with reference to the following detailed description, the features, advantages, and other aspects of the implementations of the present disclosure will become more apparent, and several implementations of the present disclosure are illustrated herein by way of example but not limitation. In the accompanying drawings:

FIG. 1 illustrates a UV/Vis transmission spectra for an intraocular lens of Examples 6A and 6B.

FIG. 2 illustrates a UV/Vis transmission spectra for an intraocular lens of Examples 6A and 6C.

FIG. 3 illustrates a UV/Vis transmission spectra for an intraocular lens of Examples 6A and 6D.

FIG. 4 illustrates a UV/Vis transmission spectra for an intraocular lens of Examples 6A and 7A-7D.

### DETAILED DESCRIPTION

Various illustrative embodiments described herein are directed to visible light absorbers, and ophthalmic devices such as ocular implant devices, contact lenses and intraocular lenses containing the visible light absorbers.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology can be applied, as long as that definition does not conflict with any other disclosure or definition applied herein or render indefinite or non-enabled any claim to which that definition is applied. To the extent that any definition or usage provided by any document incorporated herein by reference conflicts with the definition or usage provided herein, the definition or usage provided herein controls.

While compositions and processes are described in terms of “comprising” various components or steps, the compositions and processes can also “consist essentially of” or “consist of” the various components or steps, unless stated otherwise.

The terms “a,” “an,” and “the” are intended to include plural alternatives, e.g., at least one. The terms “including”, “with”, and “having”, as used herein, are defined as comprising (i.e., open language), unless specified otherwise.

Various numerical ranges are disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. For example, all numerical end points of ranges disclosed herein are approximate, unless excluded by proviso.

Values or ranges may be expressed herein as “about”, from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In another aspect, use of the term “about” means±20% of the stated value, ±15% of the stated value, ±10% of the stated value, ±5% of the stated value, ±3% of the stated value, or ±1% of the stated value.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material are 10 mol. % of component.

The term “substituted” means that a hydrogen atom in the underlying moiety is optionally replaced by a substituent. Any substituent may be used that is sterically practical at the substitution site and is synthetically feasible. Identification of a suitable optional substituent is well within the capabilities of an ordinarily skilled artisan.

Applicant reserves the right to proviso out or exclude any individual members of any such group of values or ranges, including any sub-ranges or combinations of sub-ranges within the group, that can be claimed according to a range or in any similar manner, if for any reason Applicant chooses to claim less than the full measure of the disclosure, for example, to account for a reference that Applicant may be unaware of at the time of the filing of the application. Further, Applicant reserves the right to proviso out or exclude any members of a claimed group.

As mentioned above, it has been shown that short wavelength visible light, both violet and blue, is damaging to cells both in in vitro and in vivo. Polymerizable benzotriazole, benzophenone and triazine absorbers are known. Most of these compounds are known as UV absorbers, though some may be known to also absorb some portion of visible light. Many absorbers contain ethylenically unsaturated groups, such as methacrylate, acrylate, methacrylamide, acrylamide or styrene groups. Copolymerization with other ingredients in the lens materials incorporates the absorbers into the resulting polymer chain.

Benzotriazole vinylic monomers are typically photo-stable and can absorb a large amount of both visible and UV light, but may be difficult and expensive to make. Also, they may not be soluble in a lens formulation. If the absorber does not have sufficient solubility in a lens formulation, the absorber may coalesce into domains that could interact with light and result in decreased optical clarity of the lens. Thus, there is a need for a visible light absorbing vinylic monomer that absorbs light between about 380 and about 500 nanometers (nm) wavelength, shows good solubility in lens formulations, is photo-stable, and can be made in a cost-effective manner.

The visible light absorbers described herein overcome the foregoing problems and advantageously provide an ophthalmic device exhibiting sufficient blocking of visible light between about 380 and about 500 nm wavelength. In non-limiting illustrative embodiments, a visible light absorber is represented by an azo compound having a structure of Formula I:

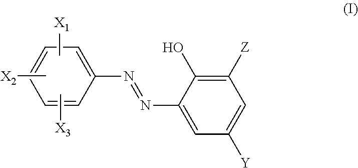

wherein X₁, X₂ and X₃ are independently hydrogen, a substituted or unsubstituted, linear or branched, alkyl group, a substituted or unsubstituted alkoxy group, a vinyl group or an allyl group; Y is hydrogen, a substituted or unsubstituted, linear or branched, alkyl group, or a vinyl group or an allyl group; and Z is hydrogen, a substituted or unsubstituted, linear or branched, alkyl group, a substituted or unsubstituted alkoxy group, a vinyl group or an allyl group, wherein at least one of X₁, X₂, X₃, Y and Z is a vinyl group or an allyl group.

Representative examples of substituted or unsubstituted alkyl groups for use herein include, by way of example, a straight or branched hydrocarbon chain radical containing carbon and hydrogen atoms of from 1 to about 18 carbon atoms or from 1 to 6 carbon atoms with or without unsaturation, to the rest of the molecule, e.g., methyl, ethyl, n-propyl, 1-methylethyl (isopropyl), n-butyl, n-pentyl, etc., and the like.

Representative examples of substituted or unsubstituted alkoxy groups for use herein include, by way of example, an alkyl group as defined herein attached via oxygen linkage to the rest of the molecule, i.e., of the general formula —OR⁴, wherein R⁴ is an alkyl, cycloalkyl, or aromatic group as defined herein, e.g., —OCH₃, —OC₂H₅, or —OC₆H₅ which may be substituted or unsubstituted, and the like.

Representative examples of cycloalkyl groups for use herein include, by way of example, a substituted or unsubstituted, non-aromatic mono or multicyclic ring system of about 3 to about 30 carbon atoms or from 3 to about 12 carbon atoms or from 3 to about 6 carbon atoms such as, for example, cyclopropyl, cyclobutyl, cyclopentyl, cyclohexyl, perhydronapththyl, adamantyl and norbornyl groups, bridged cyclic groups or sprirobicyclic groups, e.g., spiro-(4, 4)-non-2-yl and the like, optionally containing one or more heteroatoms, e.g., O and N, and the like to form a heterocycloalkyl group.

In non-limiting illustrative embodiments, X₁ and X₂ are hydrogen, X₃ is a vinyl group, Y is a C₁ to C₆ alkyl group and Z is a hydrogen.

In non-limiting illustrative embodiments, X₁ and X₂ are hydrogen, X₃ is a C₁ to C₆ alkyl group, Y is a C₁ to C₆ alkyl group and Z is an allyl group.

In non-limiting illustrative embodiments, each of X₁, X₂ and X₃ are independently a C₁ to C₆ alkyl group, Y is a vinyl group and Z is a substituted or unsubstituted alkoxy group.

In non-limiting illustrative embodiments, X₁ and X₂ are hydrogen, X₃ is a C₁ to C₆ alkyl group, Y is a vinyl group or an allyl group and Z is a C₁ to C₆ alkoxy group.

In non-limiting illustrative embodiments, each of X₁, X₂ and X₃ are independently a C₁ to C₆ alkyl group, Y is a vinyl group or an allyl group and Z is a hydrogen.

In non-limiting illustrative embodiments, one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

In some embodiments, a suitable vinyl group can be represented by the general formula:

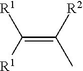

wherein R¹ is independently hydrogen or an alkyl radical having 1 to 6 carbon atoms and R² is hydrogen or an alkyl radical having 1 to 6 carbon atoms. In some embodiments, a suitable vinyl group is represented by the formula: H₂C═CH—.

In some embodiments, a suitable allyl group can be represented by the general formula:

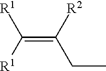

wherein R¹ is independently hydrogen or an alkyl radical having 1 to 6 carbon atoms and R² is hydrogen or an alkyl radical having 1 to 6 carbon atoms. In some embodiments, a suitable vinyl group is represented by the formula: H₂C═CHCH₂—.

Representative examples of azo compounds having a structure of Formula I include the following:

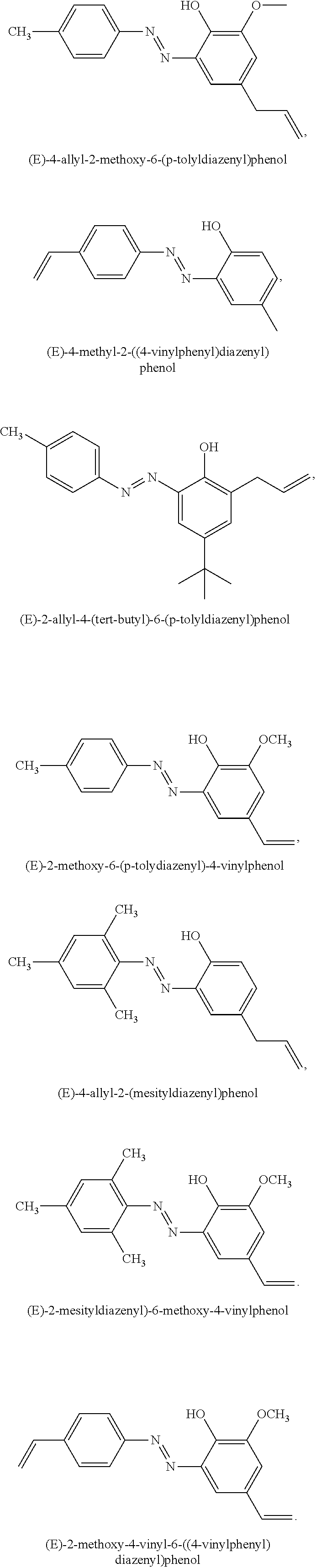

The visible light absorbing azo compounds having a structure of Formula I can be prepared using methods known in the art, and as exemplified in the examples. For example, as set forth below in Scheme I, a reactive diazonium salt of a substituted aniline derivative can be prepared in step 1, and then the reactive diazonium salt of a substituted aniline derivative is azo-coupled with a phenolic compound in step 2 to form a visible light absorbing azo compound having a structure of Formula I.

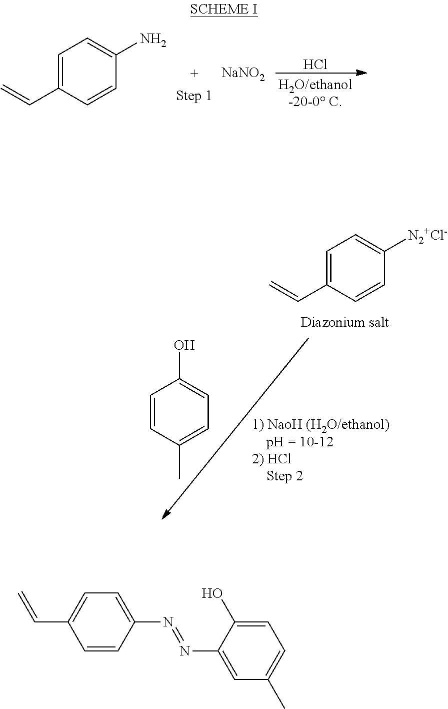

This method is merely illustrative and other methods for making the visible light absorbing azo compounds having a structure of Formula I are contemplated herein. For example, as one skilled in the art would appreciate, other azo compounds having a structure of Formula I may be prepared using analogous reaction sequences and corresponding starting materials. In general, azo compounds having a structure of Formula I may be prepared by azo coupling aniline (optionally substituted) with a variety of phenolic compounds.

In non-limiting illustrative embodiments, as may be combined with one or more of the preceding paragraphs, the visible light absorbers described herein may be incorporated in a number of materials in a variety of applications where it is desirable to block visible light such as blue light, i.e., absorbs light between about 380 and about 500 nm wavelength. In non-limiting illustrative embodiments, one application includes incorporating the visible light absorbers as described herein in ophthalmic devices.

The ophthalmic devices described herein are intended for direct contact with body tissue or body fluid. As used herein, the term “ophthalmic device” refers to devices that reside in or on the eye. These devices can provide optical correction, wound care, drug delivery, diagnostic functionality or cosmetic enhancement or effect or a combination of these properties. Suitable ophthalmic devices include, for example, ophthalmic lenses such as soft contact lenses, e.g., a soft, hydrogel lens, soft, non-hydrogel lens and the like, hard contact lenses, e.g., a hard, gas permeable lens material and the like, intraocular lenses, overlay lenses, ocular inserts, optical inserts, eyeglasses, sunglasses and the like. As is understood by one skilled in the art, a lens is considered to be “soft” if it can be folded back upon itself without breaking.

In illustrative embodiments, the ophthalmic device comprises a lens, inlay, outlay, or insert selected from an intraocular implant or lens, a contact lens, a corneal inlay, a corneal outlay, and a corneal insert.

In some illustrative embodiments, the ophthalmic device is an intraocular implant or lens. Specifically, the ophthalmic device described herein includes intraocular implants and/or lenses made at least partially or completely from the polymerized monomeric mixtures described herein. Such intraocular implants or lenses can include an optic portion and one or more haptic portions. For example, the polymerized monomeric mixtures described herein will make up part or all of the optic portion of the intraocular implant or lens. In some embodiments, the optic portion of the implant or lens will have a core made from one of the polymerized monomeric mixtures described herein surrounded by a different polymer or material. Implants or lenses in which the optic portion is made up of, at least partially, one of the polymerized monomeric mixtures described herein will usually also have a haptic portion. The haptic portion can also be made of the polymerized monomeric mixtures described herein or can be made of a different material, for example another polymer.

In some embodiments, the intraocular implant or lens is a one-piece lens having a soft, foldable central optic region and an outer peripheral region (haptic-region) in which both regions are made of the same polymer. In other embodiments, the optic and haptic regions can be formed from different types of polymers or materials, if desired. Some implants or lenses can also have haptic portions that are made up of different materials, for example where one or more haptic portions is made from the same material as the optic portion and other haptic portions are made of materials other than the polymerized monomeric mixtures described herein. Multicomponent implants or lenses can be made by embedding one material in the other, concurrent extrusion processes, solidifying the hard material about the soft material, or forming an interpenetrating network of the rigid component into a preformed hydrophobic core. In instances where one or more haptic portions are made from a different material than the optic portion of the lens, the haptic portion can be attached to the optic portion in any manner known in the art, such as by drilling a hole or holes in the optic portion and inserting the haptic portion.

The polymerized monomeric mixtures described herein have been designed so that they are capable of being folded so that the intraocular lens can be inserted into the eye of an individual through a small incision. In illustrative embodiments, that incision will be less than 2.5 mm. In other illustrative embodiments, the incision will be less than 2 mm. The haptic portion of the lens provides the required support for the implant or lens in the eye after insertion and unfolding of the lens and tends to help stabilize the position of the lens after insertion and the closure of the incision. The shape of the haptic portion design is not particularly limited and can be any desired configuration, for example, either a plate type or graduated thickness spiral filaments, also known as a C-loop design.

The optic portion of the intraocular lens can be approximately 2 to 6 mm in diameter prior to hydration. The approximate 2 to 6 mm diameter is fairly standard in the art and is generally chosen to cover the pupil in its fully dilated state under naturally occurring conditions. However, this size is not limited to any particular diameter or size of intraocular lens, and other sizes are contemplated. Furthermore, it is not necessary that the lens optic portion be circular; it could also be oval, square, or any other shape as desired.

The intraocular lens can further include one or more non-optical haptic components extending away from the outermost peripheral surface of the optic portion. The haptic components can be of any desired shape, for example, graduated spiral filaments or flat plate sections and are used to support the lens within the posterior chamber of the eye. Lenses having any desired design configuration can be fabricated. Should the intraocular lens include other components besides the optical and haptic portions, such other portions can be made of a polymer as are the haptic and optic portions, or if desired, another material.

The intraocular lenses may be inserted into the eye in any manner known in the art. For example, in one embodiment, the intraocular lens may be folded prior to insertion into the eye using an intraocular lens inserter or by small, thin forceps of the type typically used by ophthalmic surgeons. After the implant or lens is in the targeted location, it is released to unfold. As is well known in the art, typically the lens that is to be replaced is removed prior to insertion of the intraocular lens. The intraocular lens described herein can be made of a generally physiologically inert soft polymeric material that is capable of providing a clear, transparent, refractive lens body even after folding and unfolding. In some embodiments, the foldable intraocular lens can be inserted into any eye by injection whereby the mechanically compliant material is folded and forced through a small tube such as a 1 mm to 3 mm inner diameter tube.

In non-limiting illustrative embodiments, as may be combined with one or more of the preceding paragraphs, the ophthalmic devices can be obtained from a polymerization product of a monomeric mixture comprising (a) one or more visible light absorbers comprising an azo compound having a structure of Formula I, and (b) one or more ophthalmic device-forming monomers.

In some embodiments, the one or more visible light absorbers comprising an azo compound having a structure of Formula I can be present in the monomeric mixture in an amount of about 0.005 wt. % to about 0.5 wt. %, based on the total weight of the monomeric mixture. In some embodiments, the one or more visible light absorbers comprising an azo compound having a structure of Formula I can be present in the monomeric mixture in an amount of about 0.005 wt. % to about 0.2 wt. %, based on the total weight of the monomeric mixture. In some embodiments, the one or more visible light absorbers comprising an azo compound having a structure of Formula I can be present in the monomeric mixture in an amount of about 0.005 wt. % to about 0.1 wt. %, based on the total weight of the monomeric mixture. In some embodiments, the one or more visible light absorbers comprising an azo compound having a structure of Formula I can be present in the monomeric mixture in an amount of about 0.01 wt. % to about 0.04 wt. %, based on the total weight of the monomeric mixture.

In some embodiments, the one or more ophthalmic device-forming monomers can be present in the monomeric mixture in an amount ranging from about 50 wt. % to about 98 wt. %, based on the total weight of the monomeric mixture. In some embodiments, the one or more ophthalmic device-forming monomers can be present in the monomeric mixture in an amount ranging from about 90 wt. % to about 96 wt. %, based on the total weight of the monomeric mixture.

In non-limiting illustrative embodiments, as may be combined with one or more of the preceding paragraphs, the one or more ophthalmic device-forming monomers for use in the monomeric mixture can be one or more hydrophilic monomers. Suitable hydrophilic monomers include, for example, unsaturated carboxylic acids, acrylamides, vinyl lactams, hydroxyl-containing-(meth)acrylates, hydrophilic vinyl carbonates, hydrophilic vinyl carbamates, hydrophilic oxazolones, and poly(alkene glycols) functionalized with polymerizable groups and the like and mixtures thereof. Representative examples of unsaturated carboxylic acids include methacrylic acid, acrylic acid and the like and mixtures thereof. Representative examples of amides include alkylamides such as N,N-dimethylacrylamide, N,N-dimethylmethacrylamide and the like and mixtures thereof. Representative examples of cyclic lactams include N-vinyl-2-pyrrolidone, N-vinyl caprolactam, N-vinyl-2-piperidone and the like and mixtures thereof. Representative examples of hydroxyl-containing (meth)acrylates include 2-hydroxyethyl methacrylate (HEMA), glycerol methacrylate and the like and mixtures thereof. Still further examples are the hydrophilic vinyl carbonate or vinyl carbamate monomers disclosed in U.S. Pat. No. 5,070,215, and the hydrophilic oxazolone monomers disclosed in U.S. Pat. No. 4,910,277. Other suitable hydrophilic monomers will be apparent to one skilled in the art. Mixtures of the foregoing hydrophilic monomers can also be used in the monomeric mixtures herein.

In an illustrative embodiment, the hydrophilic monomers are one or more of an acrylamide and a hydroxyl-containing-(meth)acrylate as defined above.

In a non-limiting illustrative embodiment, a hydrophilic monomer for use herein can be a high glass transition temperature (Tg) hydrophilic monomer or homopolymer. A “high glass transition temperature hydrophilic monomer or homopolymer” is a hydrophilic monomer or homopolymer that, when incorporated into a polymer with one or more other monomers and cross-linkers, increases the glass transition temperature of the resulting polymer as compared to a resulting polymer formed without the high glass transition temperature hydrophilic monomer or homopolymer. In illustrative embodiments, the hydrophilic monomers and/or their corresponding homopolymer can have Tg values of greater than 50° C., e.g., Tg values between about 60 to about 100° C. For example, a hydrophilic monomer such as HEMA does not have a Tg value, however, when it is polymerized into its corresponding homopolymer (polyHEMA) then the corresponding homopolymer can have a Tg value of about 60° C. By raising the Tg value there is an added benefit of reducing surface tack which is advantageous for the fabrication of IOLs with minimal particulates and other cosmetic defects.

In some embodiments, the hydrophilic monomers can be present in the monomeric mixture in an amount ranging from about 15 wt. % to about 40 wt. %, based on the total weight of the monomeric mixture. In some embodiments, the hydrophilic monomers can be present in the monomeric mixture in an amount ranging from about 25 wt. % to about 35 wt. %, based on the total weight of the monomeric mixture.

In non-limiting illustrative embodiments, as may be combined with one or more of the preceding paragraphs, the one or more ophthalmic device-forming monomers for use in the monomeric mixture can be one or more polyethylene glycol (meth)acrylate monomers. In some embodiments, the one or more polyethylene glycol methacrylate monomers can be represented by a structure of Formula II:

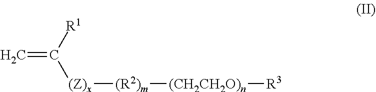

in which:
R¹ is a hydrogen atom or a methyl radical; Z is a divalent group selected from the group —COO—, —CONH—, —CONCH₃—, —OCO—, —O—, —SO₂— CO—O—CO— and —CO—CH₂—CO—; x is 0 or 1; R² is a linear, branched, cyclic, or aromatic, saturated or unsaturated, divalent carbon-based radical, of 1 to 30 carbon atoms; m is 0 or 1; n is an integer between 3 and 300 inclusive; and R³ is a hydrogen atom or a linear, branched, cyclic, or aromatic, saturated or unsaturated, carbon-based radical, of 1 to 30 carbon atoms.

In some embodiments, R² may be an alkylene radical containing 1 to 20 carbon atoms, such as methylene, ethylene, n-propylene, isopropylene, n-butylene, isobutylene, tert-butylene, pentylene, isopentylene, n-hexylene, isohexylene, heptylene, isoheptylene, n-octylene, isooctylene, nonylene, isononylene, decylene, isodecylene, n-dodecylene, isododecylene, tridecylene, n-tetradecylene, hexadecylene, n-octadecylene, docosanylene or arachinylene; a substituted or unsubstituted cycloalkylene radical containing 5 to 10 carbon atoms, such as cyclopentylene, cyclohexylene, cycloheptylene, cyclooctylene, cyclononylene or cyclodecylene; a phenylene radical —C₆H₄— (ortho, meta or para), optionally substituted with a C₁-C₁₂ alkyl radical optionally comprising 1 to 18 heteroatoms chosen from O, N, S, F, Si and P; a benzylene radical —C₆H₄—CH₂—; a radical of formula —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH—, —CH₂—CH₂—NH—CO—NH—; —CH₂CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —CH₂CH₂—CHR′—O— with R′ and R″ representing a linear or branched C₁-C₂₂ alkyl; or a mixture of these radicals.

In some embodiments, n is between 5 and 200 inclusive, or between 6 and 120 inclusive, or between 7 and 50 inclusive.

In some embodiments, R³ is a hydrogen atom; a phenyl radical optionally substituted with a C₁-C₁₂ alkyl radical; a C₁-C₃₀ or a C₁-C₂₂ or a C₂-C₁₆ alkyl radical; or a C₃-C₁₂ or a C₄-C₈ or a C₅-C₆ cycloalkyl radical. In some embodiments, R³ may be methyl, ethyl, propyl, benzyl, ethylhexyl, lauryl, stearyl and behenyl (—(CH₂)₂₁—CH₃) chains.

In some embodiments, a suitable polyethylene glycol (meth)acrylate monomer is a poly(ethylene glycol) phenyl ether acrylate of average molecular weights 236, 280 or 324, available from Aldrich.

In some embodiments, the polyethylene glycol (meth)acrylate monomers can be present in the monomeric mixture in an amount ranging from about 35 wt. % to about 60 wt. %, based on the total weight of the monomeric mixture. In some embodiments, the polyethylene glycol (meth)acrylate monomers can be present in the monomeric mixture in an amount ranging from about 40 wt. % to about 50 wt. %, based on the total weight of the monomeric mixture.

In non-limiting illustrative embodiments, as may be combined with one or more of the preceding paragraphs, the one or more ophthalmic device-forming monomers for use in the monomeric mixture can be one or more heterocyclic monomers having an ethylenically unsaturated reactive end group. Ethylenically unsaturated reactive end groups are well known to those skilled in the art. Suitable ethylenically unsaturated polymerizable groups include, for example, (meth)acrylates, vinyl carbonates, O-vinyl carbamates, N-vinyl carbamates, styrene-containing radicals and (meth)acrylamides. As used herein, the term “(meth)” denotes an optional methyl substituent. Thus, terms such as “(meth)acrylate” denotes either methacrylate or acrylate, and “(meth)acrylamide” denotes either methacrylamide or acrylamide. In one embodiment, an ethylenically unsaturated reactive end group can be represented by the general formula:

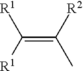

wherein R¹ is independently hydrogen, fluorine or methyl; R² is independently hydrogen, fluorine, an alkyl radical having 1 to 6 carbon atoms, or a —CO—Y—R³ radical wherein Y is-O—, —S— or —NH— and R³ is a divalent alkylene radical having 1 to about 10 carbon atoms.

In another illustrative embodiment, the (meth)acrylic group of a heterocyclic (meth)acrylic monomer is a (meth)acrylate-containing reactive end group. Suitable (meth)acrylate-containing reactive end groups can be those represented by the structure:

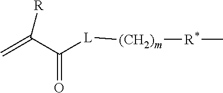

wherein R is hydrogen or methyl; L is O, NR¹, or S, where R¹ is H, CH₃, CH₂CH₃, or CH(CH₃)₂; m is an integer from 0 to 4 and R* is a linking group or bond. Suitable linking groups include, for example, any divalent hydrocarbon radical or moiety such as independently straight or branched, substituted or unsubstituted C₁-C₆ alkyl group, or an —OR² group where R² is an alkyl group from 1 to 6 carbon atoms.

The term “heterocyclic,” by itself or in combination with other terms, means, unless otherwise stated, a non-aromatic cyclic version of “alkyl” and “heteroalkyl,” respectively, wherein the carbons making up the ring or rings do not necessarily need to be bonded to a hydrogen due to all carbon valencies participating in bonds with non-hydrogen atoms. Representative examples of heterocyclic ring groups of the heterocyclic monomer having an ethylenically unsaturated reactive end group for use herein include, by way of example, a substituted or unsubstituted stable 3 to about 15 membered ring radical, containing carbon atoms and from one to five heteroatoms, e.g., nitrogen, phosphorus, oxygen, sulfur and mixtures thereof. Suitable heterocyclic ring radicals of the heterocyclic monomer having an ethylenically unsaturated reactive end group for use herein may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states. Examples of such heterocyclic ring radicals include, but are not limited to, azetidinyl, carbazolyl, dioxolanyl, piperidinyl, piperazinyl, pyrrolidinyl, 2-oxopiperazinyl, 2-oxopiperidinyl, 2-oxopyrrolidinyl, morpholinyl, and the like and mixtures thereof.

In an illustrative embodiment, a heterocyclic moiety may include one ring heteroatom (e.g., O, N, S or P). In an illustrative embodiment, a heterocycloalkyl moiety may include two optionally different ring heteroatoms (e.g., O, N, S, Si, or P).

The heterocyclic monomers having an ethylenically unsaturated reactive end group can be unsubstituted or substituted. In an illustrative embodiment, a substituted heterocyclic monomer having an ethylenically unsaturated reactive end group include the heterocyclic ring portion of the heterocyclic monomer being substituted with one or more of a hydroxy group, halogen group, carboxyl group, cyano group, nitro group, oxo (═O) group, thio (═S) group, a substituted or unsubstituted alkyl group, or a substituted or unsubstituted alkoxy group.

In a non-limiting illustrative embodiment, a heterocyclic (meth)acrylic monomer can be represented by a structure of Formula III:

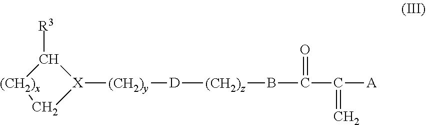

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃) 2; D is O, S, or a bond; A is H or CH₃; and z is 0 to 4, provided that if D is a bond then at least one of y and z is 0.

In an illustrative embodiment, the one or more heterocyclic (meth)acrylic monomers are represented by a structure of Formula III, wherein x is an integer from 1 to 4; y is 0; R³ is an oxo group, X is N; D is O; z is an integer from 1 to 4; B is O; and A is H or CH₃.

In an illustrative embodiment, the one or more heterocyclic (meth)acrylic monomers are represented by a structure of Formula III, wherein x is an integer from 1 to 3; y is 0; R³ is an oxo group, X is N; D is O; z is an integer from 2 to 3; B is O; and A is H or CH₃.

In an illustrative embodiment, the one or more heterocyclic (meth)acrylic monomers are represented by a structure of Formula III, wherein x is an integer from 1 or 2; y is an integer from 1 to 4; R³ is an oxo group, X is N, D is a bond; z is 0; B is O; and A is H or CH₃.

In an illustrative embodiment, suitable one or more heterocyclic (meth)acrylic monomers include, for example, 3-(2-oxopyrrolidin-1-yl) propyl acrylate, 2-(2-oxopyrrolidin-1-yl)ethyl acrylate, 3-(2-oxopyrrolidin-1-yl) propyl methacrylate, 2-(2-oxopyrrolidin-1-yl)ethyl methacrylate, (2-oxopyrrolidin-1-yl)methyl methacrylate, 4-(2-oxopyrrolidin-1-yl)butyl methacrylate, (2-oxopyrrolidin-1-yl)methyl acrylate, 4-(2-oxopyrrolidin-1-yl)butyl acrylate, 3-(1H-pyrrol-1-yl) propyl acrylate), 3-(1H-pyrrol-1-yl) propyl methacrylate and the like and mixtures thereof.

In some embodiments, the heterocyclic monomers having an ethylenically unsaturated reactive end group can be present in the monomeric mixture in an amount ranging from about 15 wt. % to about 50 wt. %, based on the total weight of the monomeric mixture. In some embodiments, the heterocyclic monomers having an ethylenically unsaturated reactive end group can be present in the monomeric mixture in an amount ranging from about 25 wt. % to about 40 wt. %, based on the total weight of the monomeric mixture.

In non-limiting illustrative embodiments, as may be combined with one or more of the preceding paragraphs, the one or more ophthalmic device-forming monomers for use in the monomeric mixture can be one or more aromatic monomers having an ethylenically unsaturated reactive end group. The term “aromatic” as used herein refers to a conjugated cyclic unsaturated hydrocarbon typified by the benzene skeleton, the naphthalene skeleton or the like in a molecule. Representative examples of aromatic groups of the aromatic monomers having an ethylenically unsaturated reactive end group for use herein include, by way of example, a substituted or unsubstituted monoaromatic or polyaromatic radical containing from about 5 to about 25 carbon atoms or from 6 to 12 carbon atoms or from 6 to 10 carbon atoms such as, for example, phenyl, naphthyl, tetrahydronapthyl, indenyl, biphenyl and the like, optionally containing one or more heteroatoms, e.g., O and N, and the like.

In a non-limiting illustrative embodiment, an aromatic monomer having an ethylenically unsaturated reactive end group can be represented by a structure of Formula IV:

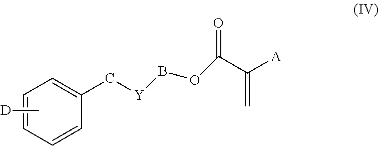

wherein A is H, CH₃, CH₂CH₃, or CH₂OH;
B is (CH₂)ₘ or [0 (CH₂) 2]z;
Cis (CH₂) w;
m is 2-6;
z is 1-10;
Y is nothing, O, S, or NR, provided that if Y is O, S, or NR then B is (CH₂)ₘ;
R′ is H, CH₃, Cₙ′H₂n′+1 (n′=1−10), iso-OC₃H₇, C₆H₅, or CH₂C₆H₅; w is 0-6, provided that m+w≤8; and
D is H, C₁-C₄ alkyl, C₁-C₄ alkoxy, C₆H₅, CH₂C₆H₅ or halogen.

In non-limiting illustrative embodiments, as may be combined with one or more of the preceding paragraphs, the aromatic monomers having an ethylenically unsaturated reactive end group can be one or more arylalkyl monomers having an ethylenically unsaturated reactive end group.

Representative examples of arylalkyl groups for use herein include, by way of example, a substituted or unsubstituted aromatic group as defined above directly bonded to an alkyl group, e.g., —CH₂C₆H₅, —C₂H₅C₆H₅ and the like.

The ethylenically unsaturated reactive end group of the aromatic monomers having an ethylenically unsaturated reactive end group can be any of those discussed above. In an illustrative embodiment, an ethylenically unsaturated reactive end group is a (meth)acrylate end group.

Specific examples thereof of an aromatic (meth)acrylic monomer include 2-phenylethyl acrylate, 2-phenylethyl methacrylate, 2-phenoxyethyl acrylate, 2-phenoxyethyl methacrylate, 3-phenylpropyl acrylate, 3-phenylpropyl methacrylate and the like and mixtures thereof.

In some embodiments, the aromatic monomers having an ethylenically unsaturated reactive end group can be present in the monomeric mixture in an amount ranging from about 40 wt. % to about 80 wt. %, based on the total weight of the monomeric mixture. In some embodiments, the aromatic monomers having an ethylenically unsaturated reactive end group can be present in the monomeric mixture in an amount ranging from about 50 wt. % to about 70 wt. %, based on the total weight of the monomeric mixture.

In non-limiting illustrative embodiments, as may be combined with one or more of the preceding paragraphs, representative examples of one or more ophthalmic device-forming monomers for use in the monomeric mixture include methyl methacrylate, 2-hydroxyethyl acrylate, 2-hydroxyethyl methacrylate, 3-hydroxypropyl acrylate, 3-hydroxypropyl methacrylate, n-vinyl pyrolidone, styrene, eugenol (4-hydroxyvinylbenzene), α-methylstyrene. In addition, for high-refractive index foldable lens applications, suitable monomers include, but are not limited to 2-ethylphenoxy methacrylate, 2-ethylphenoxy acrylate, 2-ethylthiophenyl methacrylate, 2-ethylthiophenylacrylate, 2-ethylaminophenyl methacrylate, phenyl methacrylate, benzyl methacrylate, 2-phenylethyl methacrylate (PEMA), 2-phenylethyl acrylate (PEA), 3-phenylpropyl methacrylate, 4-phenylbutyl methacrylate, 4-methylphenyl methacrylate, 4-methylbenzyl methacrylate, 2-2-methylphenylethyl methacrylate, 2-3-methylphenylethyl methacrylate, 2-4-methylphenylethyl methacrylate, 2-(4-propylphenyl)ethyl methacrylate, 2-(4-(1-methylethyl)phenyl)ethyl methacrylate, 2-(4-methoxyphenyl)ethyl methacrylate, 2-(4-cyclohexylphenyl)ethyl methacrylate, 2-(2-chlorophenyl)ethyl methacrylate, 2-(3-chlorophenyl)ethyl methacrylate, 2-(4-chlorophenyl)ethyl methacrylate, 2-(4-bromophenyl)ethyl methacrylate, 2-(3-phenylphenyl)ethyl methacrylate, 2-(4-phenylphenyl)ethyl methacrylate), 2-(4-benzylphenyl)ethyl methacrylate, and the like, including the corresponding methacrylates and acrylates. N-vinyl pyrolidone, styrene, eugenol and α-methyl styrene may also be suitable for high-refractive index foldable lens applications.

In non-limiting illustrative embodiments, as may be combined with one or more of the preceding paragraphs, the monomeric mixtures may further comprise one or more crosslinking agents. Suitable crosslinking agents for use herein are known in the art. For example, in non-limiting illustrative embodiments, suitable one or more cross-linking agents include one or more crosslinking agents containing at least two ethylenically unsaturated reactive end groups. In one embodiment, the ethylenically unsaturated reactive end groups are (meth)acrylate-containing reactive end groups. In another embodiment, the ethylenically unsaturated reactive end groups are non-(meth)acrylate reactive end groups. In one embodiment, the ethylenically unsaturated reactive end groups are a combination of one or more (meth)acrylate-containing reactive end groups and one or more non-(meth)acrylate reactive end groups.

In an illustrative embodiment, suitable one or more crosslinking agents containing at least two ethylenically unsaturated reactive end groups include, for example, one or more di-, tri- or tetra(meth)acrylate-containing crosslinking agents. In an illustrative embodiment, suitable one or more di-, tri- or tetra(meth)acrylate-containing crosslinking agents include, for example, alkanepolyol di-, tri- or tetra(meth)acrylate-containing crosslinking agents such as, for example, one or more alkylene glycol di(meth)acrylate crosslinking agents, one or more alkylene glycol tri(meth)acrylate crosslinking agents, one or more alkylene glycol tetra(meth)acrylate crosslinking agents, one or more alkanediol di(meth)acrylate crosslinking agents, alkanediol tri(meth)acrylate crosslinking agents, alkanediol tetra(meth)acrylate crosslinking agents, agents, one or more alkanetriol di(meth)acrylate crosslinking agents, alkanetriol tri(meth)acrylate crosslinking agents, alkanetriol tetra(meth)acrylate crosslinking agents, agents, one or more alkanetetraol di(meth)acrylate crosslinking agents, alkanetetraol tri(meth)acrylate crosslinking agents, alkanetetraol tetra(meth)acrylate crosslinking agents and the like and mixtures thereof.

In an illustrative embodiment, one or more alkylene glycol di(meth)acrylate crosslinking agents include tetraethylene glycol dimethacrylate, ethylene glycol di(meth)acrylates having up to about 10 ethylene glycol repeating units, butyleneglycol di(meth)acrylate and the like. In one embodiment, one or more alkanediol di(meth)acrylate crosslinking agents include butanediol di(meth)acrylate crosslinking agents, hexanediol di(meth)acrylate and the like. In one embodiment, one or more alkanetriol tri(meth)acrylate crosslinking agents are trimethylol propane trimethacrylate crosslinking agents. In one embodiment, one or more alkanetetraol tetra(meth)acrylate crosslinking agents are pentaerythritol tetramethacrylate crosslinking agents.

In a non-limiting illustrative embodiment, suitable crosslinking agents include, for example, ethylene glycol diacrylate, diethylene glycol diacrylate, allyl acrylate, 1,3-propanediol diacrylate, 2,3-propanediol diacrylate, 1,6-hexanediol diacrylate, 1,4-butanediol diacrylate, triethylene glycol diacrylate, cyclohexane-1,1-diyldimethanol diacrylate, 1,4-cyclohexanediol diacrylate, 1,3-adamantanediol diacrylate, 1,3-adamantanedimethyl diacrylate, 2,2-diethyl-1,3-propanediol diacrylate, 2,2-diisobutyl-1,3-propanediol diacrylate, 1,3-cyclohexanedimethyl diacrylate, 1,4-cyclohexanedimethyl diacrylate; neopentyl glycol diacrylate, tetraethyleneglycol diacrylate, polyethyleneglycol diacrylate; and their corresponding methacrylates.

In a non-limiting illustrative embodiment, suitable crosslinking agents include, for example, 1,4-butanediol diacrylate, 1,4-butanediol dimethacrylate, ethylene glycol dimethacrylate, diethylene glycol diacrylate, diethylene glycol dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate (Mn=700 Daltons), poly(ethylene glycol) dimethacrylate (Mn=700 Daltons), and poly(ethylene glycol) dimethacrylate (Mn=1000 Daltons).

In non-limiting illustrative embodiments, as may be combined with one or more of the preceding paragraphs, the one or more crosslinking agents can be present in the monomeric mixture in an ophthalmic device-forming amount. In some embodiments, the one or more crosslinking agents are present in the monomeric mixture in an amount of about 1 wt. % to about 10 wt. %, based on the total weight of the monomeric mixture. In some embodiments, the one or more crosslinking agents can be present in the monomeric mixture in an amount of about 3 wt. % to about 5 wt. %, based on the total weight of the monomeric mixture.

In non-limiting illustrative embodiments, as may be combined with one or more of the preceding paragraphs, the monomeric mixture can further contain a reactive (polymerizable) ultraviolet (UV) absorber in order that the ophthalmic devices such as a lens may have an ultraviolet absorbance approximately equivalent to that of the natural lens of the eye. The ultraviolet absorbing material can be any compound which absorbs ultraviolet light, i.e., light having a wavelength shorter than about 400 nm, but does not absorb any substantial amount of visible light. The use of a reactive (polymerizable) ultraviolet (UV) absorber is preferred so that the ultraviolet absorber is copolymerizable with the one or more ophthalmic device-forming monomers and is thereby covalently bound to the polymer matrix. In this way, any possible leaching of the ultraviolet absorber out of the lens and into the interior of the eye is minimized. Suitable reactive ultraviolet absorbers can be any known reactive ultraviolet absorber. In non-limiting illustrative embodiments, suitable reactive ultraviolet absorbers include, for example, 2-(2′-hydroxy-3′-methallyl-5′-methylphenyl)benzotriazole, commercially available as o-methallyl Tinuvin P (“oMTP”) from Polysciences, Inc., Warrington, Pa., 3-(2H-benzo[d][1,2,3]triazol-2-yl)-4-hydroxyphenylethyl methacrylate, and 2-(3-(tert-butyl)-4-hydroxy-5-(5-methoxy-2H-benzo[d][1,2,3]triazol-2-yl) phenoxy)ethyl methacrylate.

In some illustrative embodiments, suitable reactive ultraviolet absorbers include, for example, UV absorbers having a structure shown in Formula V.

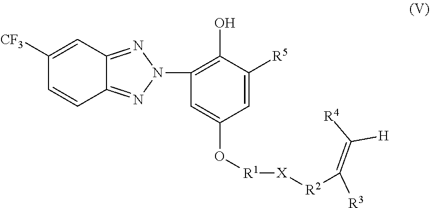

wherein:
R¹ is a C₁ to C₁₂ alkylene, (CH₂CH₂O)ₙ, (CH₂CH(CH₃)O)ₙ, or CH₂CH₂CH₂(Si(CH₃)₂O)_{b}Si(CH₃)₂CH₂CH₂CH₂;
X is a bond if R¹ is (CH₂CH₂O)ₙ or (CH₂CH(CH₃)O)ₙ, or if R² is Si(CH₃)₂, otherwise X is O, NR, or S;
R is H, CH₂Si(CH₃)₃, C₁ to C₆ alkyl, or phenyl;
R² is a bond, C(═O), C(═O)CⱼH₂ⱼ, C₁ to C₆ alkylene, phenyl, C₁ to C₆ alkylphenyl, or Si(CH₃)₂;
R³ is H or CH₃;
R⁴ is H, C₁ to C₆ alkyl, or phenyl;
R⁵═C₄ to C₁₂ t-alkyl;
b is 1 to 9;
n is 1 to 10; and
j is 1 to 6.

In some illustrative embodiments, suitable reactive ultraviolet absorbers include, for example, one or more compounds of the following formulae:

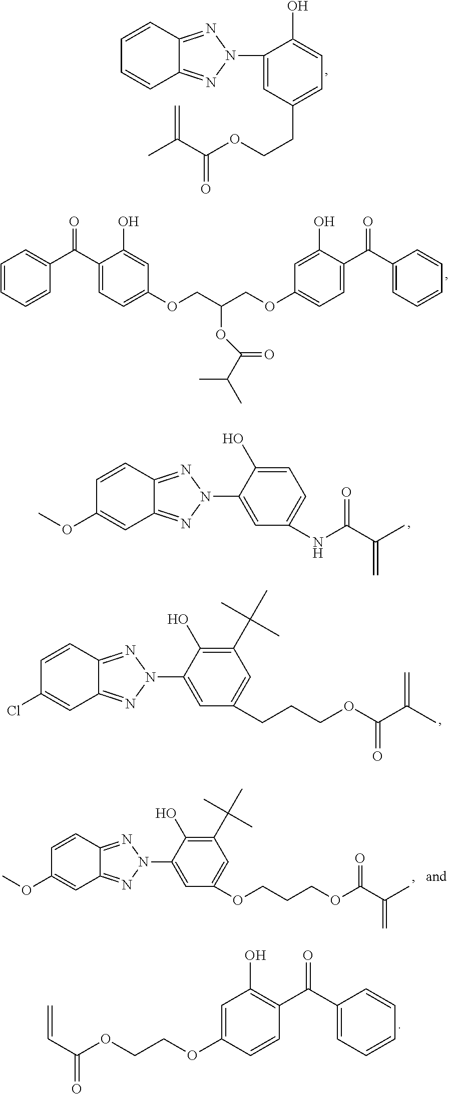

These reactive ultraviolet absorbers are merely illustrative and not intended to be limiting. Any known reactive ultraviolet absorbers or later developed reactive ultraviolet absorber is contemplated for use herein.

In some embodiments, the reactive ultraviolet absorbers can be present in the monomeric mixture in an amount ranging from about 0.1 wt. % to about 5 wt. %, based on the total weight of the monomeric mixture. In some embodiments, the reactive ultraviolet absorbers can be present in the monomeric mixture in an amount ranging from about 1.5 wt. % to about 2.5 wt. %, based on the total weight of the monomeric mixture. In some embodiments, the reactive ultraviolet absorbers can be present in the monomeric mixture in an amount ranging from about 1.5 wt. % to about 2 wt. %, based on the total weight of the monomeric mixture.

The ophthalmic devices of the illustrative embodiments described herein, e.g., intraocular lenses, can be prepared by polymerizing the foregoing monomeric mixtures to form a product that can be subsequently formed into the appropriate shape by, for example, lathing, injection molding, compression molding, cutting and the like. For example, the ophthalmic devices described herein can be prepared by combining the one or more cycloaliphatic (meth)acrylic monomers, one or more hydrophilic monomers; and one or more crosslinking agents and polymerizing the resulting mixture.

Polymerization may be facilitated by exposing the mixture to heat and/or radiation, such as ultraviolet light, visible light, or high energy radiation. A polymerization initiator may be included in the mixture to facilitate the polymerization step. Suitable polymerization initiators include thermal initiators and photoinitiators. Suitable free radical thermal polymerization initiators include, for example, organic peroxides such as acetyl peroxide, lauroyl peroxide, decanoyl peroxide, stearoyl peroxide, benzoyl peroxide, tertiarylbutyl peroxypivalate, peroxydicarbonate, and the like. Suitable free radical thermal polymerization initiators also include, for example, 2,2′-azobis(4-methoxy-2,4-dimethylvaleronitrile) (VAZO 33), 2,2′-Azobis[2-(2-imidazolin-2-yl)propane]dihydrochloride (VAZO 44), 2,2′-azobis(2-amidinopropane) dihydrochloride (VAZO 50), 2,2′-azobis(2,4-dimethylvaleronitrile) (VAZO 52), 2,2′-azobis(isobutyronitrile) (VAZO 64 or AIBN), 2,2′-azobis-2-methylbutyronitrile (VAZO 67), 1,1-azobis(1-cyclohexanecarbonitrile) (VAZO 88); 2,2′-azobis(2-cyclopropylpropionitrile), 2,2′-azobis(methylisobutyrate), 4,4′-Azobis(4-cyanovaleric acid), and combinations thereof. In one embodiment, a thermal initiator is 2,2′-azobis(isobutyronitrile) (VAZO 64 or AIBN).

Representative UV initiators are those known in the art and include benzoin methyl ether, benzoin ethyl ether, Darocure® 1173, 1164, 2273, 1116, 2959, 3331 (EM Industries) and Irgacure® 184, 651 and 819 (Ciba-Geigy), and the like.

In illustrative embodiments, the initiator will be employed in the monomeric mixture at a concentration of about 0.01 wt. % to about 5 wt. %, based on the total weight of the monomeric mixture. In other illustrative embodiments, the initiator will be employed in the monomeric mixture at a concentration of about 0.01 wt. % to about 3 wt. %, based on the total weight of the monomeric mixture. In other illustrative embodiments, the initiator will be employed in the monomeric mixture at a concentration of about 0.01 wt. % to about 1.5 wt. %, based on the total weight of the monomeric mixture.

Generally, polymerization can be carried out for about 15 minutes to about 72 hours, and under an inert atmosphere of, for example, nitrogen or argon. If thermoplastic molds made from various types of polymer resins are used, the molds can be treated with an inert gas, such as nitrogen or argon, prior to use. The resulting crude polymerization product can be extracted using an organic solvent, such as for example, acetone or isopropyl alcohol to remove unreacted components or side products which typically form during free radical polymerizations. If desired, the resulting polymerization product can be dried under vacuum, e.g., for about 5 to about 72 hours to remove residual solvent. The final product can be packaged as a dry lens or left in an aqueous solution in the final packaging configuration.

If necessary, it may be desirable to remove residual diluent from the lens before edge-finishing operations which can be accomplished by evaporation at or near ambient pressure or under vacuum. An elevated temperature can be employed to shorten the time necessary to evaporate the diluent. The time, temperature and pressure conditions for the solvent removal step will vary depending on such factors as the volatility of the diluent and the specific monomeric components, as can be readily determined by one skilled in the art. If desired, the mixture used to produce the hydrogel lens may further include crosslinking and wetting agents known in the prior art for making hydrogel materials.

The ophthalmic devices such as intraocular lenses obtained herein may be subjected to optional machining operations. For example, the optional machining steps may include buffing or polishing a lens edge and/or surface. Generally, such machining processes may be performed before or after the product is released from a mold part, e.g., the lens is dry released from the mold by employing vacuum tweezers to lift the lens from the mold, after which the lens is transferred by means of mechanical tweezers to a second set of vacuum tweezers and placed against a rotating surface to smooth the surface or edges. The lens may then be turned over in order to machine the other side of the lens.

The lens may then be transferred to individual lens packages containing a buffered saline solution. The saline solution may be added to the package either before or after transfer of the lens. Appropriate packaging designs and materials are known in the art. A plastic package is releasably sealed with a film. Suitable sealing films are known in the art and include foils, polymer films and mixtures thereof. The sealed packages containing the lenses are then sterilized to ensure a sterile product. Suitable sterilization means and conditions are known in the art and include, for example, autoclaving.

As one skilled in the art will readily appreciate, other steps may be included in the molding and packaging process described above. Such other steps can include, for example, coating the formed lens, surface treating the lens during formation (e.g., via mold transfer), inspecting the lens, discarding defective lenses, cleaning the mold halves, reusing the mold halves, and the like and combinations thereof.

The following examples are provided to enable one skilled in the art to practice the invention and are merely illustrative. The examples should not be read as limiting the scope of the invention as defined in the claims.

Various polymerization products were formed as discussed below and characterized by standard testing procedures such as:
Water %: Two sets of lenses or films were solvent extracted and vacuum dried at 70-90° C. for a minimum of 16 hours are weighed to determine the dry weight. Samples are subsequently equilibrated in deionized water at ambient temperature (20-25° C.) for a minimum of 16 hours. Samples are then blotted dry on a piece of lens paper to remove excess water, and then weighed to determine the wet weight. The percent water is calculated from the wet and dry weights as follows: Water %=100*[(wet weight)−(dry weight)]/(wet weight).
Extractable %—Two sets of six lenses or films were individually weighed after polymerization to determine the “non-extracted weight”. Samples were subsequently solvent extracted for a minimum of 16 hours to remove residual monomers, oligomers, and other impurities. Samples were then vacuum dried at 70-90° C. for a minimum of 16 hours and weighed to determine the dry weight (“extracted weight”). Extractable %=100*[(non-extracted weight)−(extracted weight)]/(non-extracted weight)

In the examples, the following abbreviations are used.
PEG-PEA: Poly(ethylene glycol)phenyl ether acrylate (average Mn=324).
HEMA: 2-hydroxyethylmethacrylate.
EGDMA: Ethylene glycol dimethacrylate.
X-monomer: 3-(3-(tert-butyl)-5-(5-chloro-2H-benzo[d][1,2,3]triazol-2-yl)-4-hydroxyphenoxy) propyl methacrylate.

### Example 1

Preparation of (E)-4-allyl-2-methoxy-6-(p-tolyldiazenyl) phenol represented by the following structure.

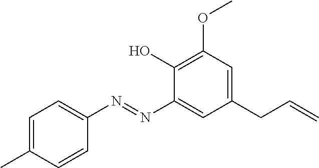 [SMILES: C=CCc1cc(/N=N/c2ccc(C)cc2)c(O)c(OC)c1]

In a 500 ml round bottom flask equipped with a magnetic stirrer was added 25.0 g (234 mmol) p-toluidine (Oakwood Chemical), 100 ml concentrated HCl (aq) (37-38%, Oakwood Chemical), 150 ml absolute ethanol, and 100 mL deionized water. Sodium nitrite (17.1 g, 247 mmol, EMD) in 100 ml water was added dropwise over 30 minutes while keeping the reaction mixture at −10° C. The reaction mixture was stirred for an additional 1 hour. Sulfamic acid (1.7 g, Oakwood Chemical) was added and the mixture was stirred for an additional 20 minutes. The solids were filtered and the cold filtrate was set aside. In a 1 L round bottom flask equipped with a magnetic stirrer was added 38.3 g (233 mmol) 4-allyl-2-methoxyphenol (Oakwood Chemical), 100 ml deionized water, and 100 ml absolute ethanol. Approximately one-fourth by volume of a solution comprised of 56.0 g (1.40 mol) NaOH in water was added to the 4-allyl-2-methoxyphenol solution and the mixture was cooled to 0° C. The diazonium mixture and remaining NaOH solution were added dropwise and simultaneously to the 4-allyl-2-methoxyphenol mixture over 30 to 60 minutes and then stirred for 1 hour at 0° C. and 20 hours at ambient temperature. The mixture was poured into 3 L deionized water and acidified to pH 4 with 1 N HCl. The solid was filtered and washed with several liters water and vacuum dried at 50° C. to give 39 g (59%) of a dark red solid.

### Example 2

Preparation of (E)-2-(p-tolyldiazenyl)-4-vinylphenol represented by the following structure.

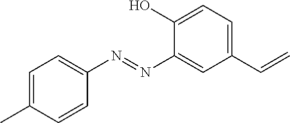 [SMILES: C=Cc1ccc(O)c(/N=N/c2ccc(C)cc2)c1]

In a 500 ml round bottom flask equipped with a magnetic stirrer was added 2.23 g (20.8 mmol) p-toluidine (Oakwood Chemical), 9.0 ml conc. HCl (aq) (37-38%, Oakwood Chemical), 100 ml absolute ethanol and 100 ml deionized water. Sodium nitrite (1.53 g, 22.2 mmol, EMD) in 30 ml water was added dropwise over 30 minutes while keeping the reaction mixture at −10° C. The reaction mixture was stirred for an additional 1 hour. Sulfamic acid (315 mg, Oakwood Chemical) was added and then stirred for an additional 20 minutes. The solids were filtered out and the cold diazonium solution was set aside. In a 1 L round bottom flask equipped with a magnetic stirrer was added 26.2 g (21.8 mmol) 4-vinylphenol (10% in propylene glycol, Oakwood Chemical), 100 ml 2-propanol, and 100 mL deionized water. Approximately one-fourth by volume of a solution comprised of 5.06 g (127 mmol) NaOH in 50 ml deionized water was added to the 4-vinylphenol solution and the reaction mixture was cooled to 0° C. The diazonium mixture and remaining NaOH solution were added simultaneously to the 4-vinylphenol mixture over 30 to 60 minutes and then stirred for an additional 1 hour at 0° C. and 20 hours at ambient temperature. The mixture was poured into 3 L deionized water and acidified to pH 4 with 1 N HCl. The solid was filtered and washed with several liters water and vacuum dried at 50° C. to give 3.1 g (63%) of a dark orange solid.

### Example 3

Preparation of (E)-4-methyl-2-((4-vinylphenyl)diazenyl) phenol represented by the following structure.

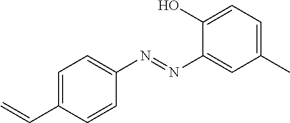 [SMILES: C=Cc1ccc(/N=N/c2cc(C)ccc2O)cc1]

In a 500 ml round bottom flask equipped with a magnetic stirrer was added 6.35 g (53.3 mmol) 4-vinylaniline (containing 0.5% KOH pellets, Oakwood Chemical), 18 ml conc. HCl (aq) (37-38%, Oakwood Chemical), 100 ml absolute ethanol and 100 ml deionized water. Sodium nitrite (3.91 g, 56.7 mmol, EMD) in 50 ml water was added dropwise over 30 minutes while keeping the reaction mixture at −10° C. The reaction mixture was stirred for an additional 1 hour. Sulfamic acid (430 mg, Oakwood Chemical) was added and then stirred for an additional 20 minutes. The solids were filtered out and a cold diazonium solution was set aside. In a 1 L round bottom flask equipped with a magnetic stirrer was added 5.48 g (50.7 mmol) p-cresol (Sigma-Aldrich), 100 ml 2-propanol, and 100 mL deionized water. Approximately one-fourth by volume of a solution comprised of 12.8 g (320 mmol) NaOH in 80 ml deionized water was added to the p-cresol solution and the reaction mixture was cooled to 0° C. The diazonium mixture and remaining NaOH solution were added simultaneously to the p-cresol mixture over 30 to 60 minutes and then stirred for an additional 1 hour at 0° C. and 20 hours at ambient temperature. The mixture was poured into 3 L deionized water and acidified to pH 4 with 1 N HCl. The solid was filtered and washed with several liters water and vacuum dried at 50° C. to give 6 g (50%) of a dark orange solid.

### Example 4

Preparation of (E)-2-allyl-4-(tert-butyl)-6-(p-tolyldiazenyl) phenol represented by the following structure.

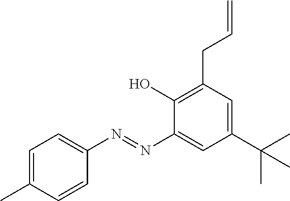 [SMILES: C=CCc1cc(C(C)(C)C)cc(/N=N/c2ccc(C)cc2)c1O]

In a 250 mL round bottom flask equipped with a magnetic stirrer was added 63.9 mg (0.596 mmol) p-toluidine (Oakwood Chemical), 0.25 ml conc. HCl (aq) (37-38%, Oakwood Chemical), 50 ml absolute ethanol and 50 ml deionized water. Sodium nitrite (40.6 mg, 0.588 mmol, EMD) in 10 ml water was added dropwise over 30 minutes while keeping the reaction mixture at −10° C. The reaction mixture was stirred for an additional 1 hour. Sulfamic acid (53 mg, Oakwood Chemical) was added and then stirred for an additional 20 minutes. The solids were filtered out and the cold diazonium solution was set aside. In a 250 mL round bottom flask equipped with a magnetic stirrer was added 94 mg (490 mmol) 2-allyl-4-tert-butyl-phenol (Sigma-Aldrich), 50 ml 2-propanol, and 50 mL deionized water. Approximately one-fourth by volume of a solution comprised of 265 mg (6.63 mmol) NaOH in 40 ml deionized water was added to the 2-allyl-4-tert-butyl-phenol solution and the reaction mixture was cooled to 0° C. The diazonium mixture and remaining NaOH solution were added simultaneously to the 2-allyl-4-tert-butyl-phenol mixture over 30 to 60 minutes and then stirred for an additional 1 hour at 0° C. and 20 hours at ambient temperature. The mixture was poured into 2 L deionized water and acidified to pH 4 with 1 N HCl. The solid was filtered and washed with several liters water and vacuum dried at 50° C. to give 100 mg (66%) of a dark red solid.

### Example 5

Preparation of (E)-4-allyl-2-(mesityldiazenyl) phenol represented by the following structure.

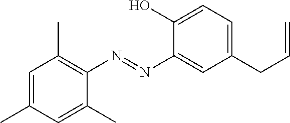 [SMILES: C=CCc1ccc(O)c(/N=N/c2c(C)cc(C)cc2C)c1]

In a 250 mL round bottom flask equipped with a magnetic stirrer was added 1.01 g (7.47 mmol) 2,4,6-trimethylaniline (Oakwood Chemical), 3 ml conc. HCl (aq) (37-38%, Oakwood Chemical), 70 ml absolute ethanol and 70 ml deionized water. Sodium nitrite (0.55 g, 8.0 mmol, EMD) in 50 ml water was added dropwise over 30 minutes while keeping the reaction mixture at −10° C. The reaction mixture was stirred for an additional 1 hour. Sulfamic acid (183 mg, Oakwood Chemical) was added and then stirred for an additional 20 minutes. The solids were filtered out and the cold diazonium solution was set aside. In a 500 mL round bottom flask equipped with a magnetic stirrer was added 1.00 g (7.45 mmol) 4-allylphenol (Ambeed), 80 ml 2-propanol, and 80 mL deionized water. Approximately one-fourth by volume of a solution comprised of 1.78 g (44.5 mmol) NaOH in 80 ml deionized water was added to the 4-allylphenol solution and the reaction mixture was cooled to 0° C. The diazonium mixture and remaining NaOH solution were added simultaneously to the 4-allylphenol mixture over 30 to 60 minutes and then stirred for an additional 1 hour at 0° C. and 20 hours at ambient temperature. The mixture was poured into 4 L deionized water and acidified to pH 4 with 1 N HCl. The solid was filtered and washed with several liters water and vacuum dried at 50° C. to give 1.2 g (57%) of a red solid.

### Examples 6A-6D

An IOL formulation was prepared from a monomeric mix made by mixing the following components, listed in Table 1 at amounts per wt. %, based on the total weight of the monomeric mixture.

TABLE 1

| Formulation | Ex. 6A | Ex. 6B | Ex. 6C | Ex. 6D |
|---|---|---|---|---|
| PEG-PEA | 43.71 | 43.70 | 43.66 | 43.57 |
| HEMA | 29.83 | 29.76 | 29.71 | 29.69 |
| Styrene | 21.85 | 21.97 | 21.90 | 21.93 |
| EGDMA | 3.96 | 3.96 | 3.93 | 4.07 |
| X-Monomer | 0.30 | 0.30 | 0.30 | 0.30 |
| Irgacure 819 | 0.35 | 0.27 | 0.46 | 0.41 |
| Blue blocker of | — | 0.036 | — | — |
| Example 3 | | | | |
| Blue blocker of | — | — | 0.042 | — |
| Example 1 | | | | |
| Blue blocker of | — | — | — | 0.041 |
| Example 4 | | | | |

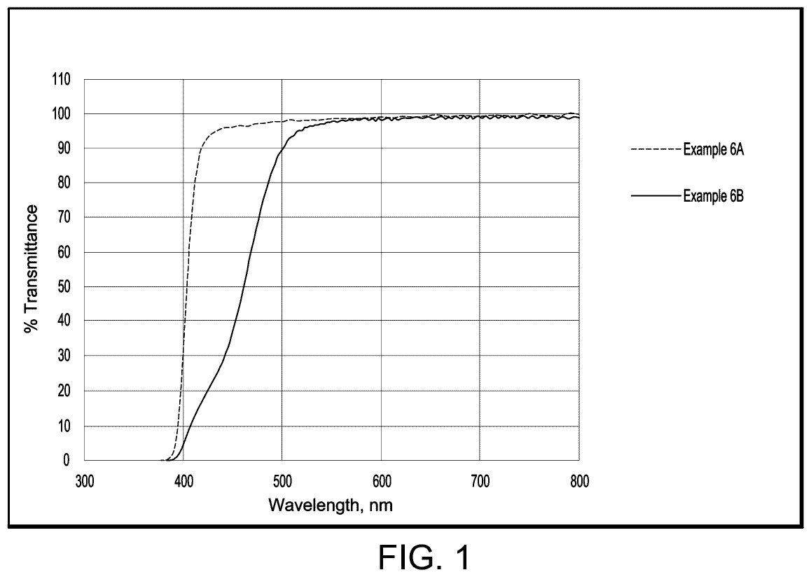

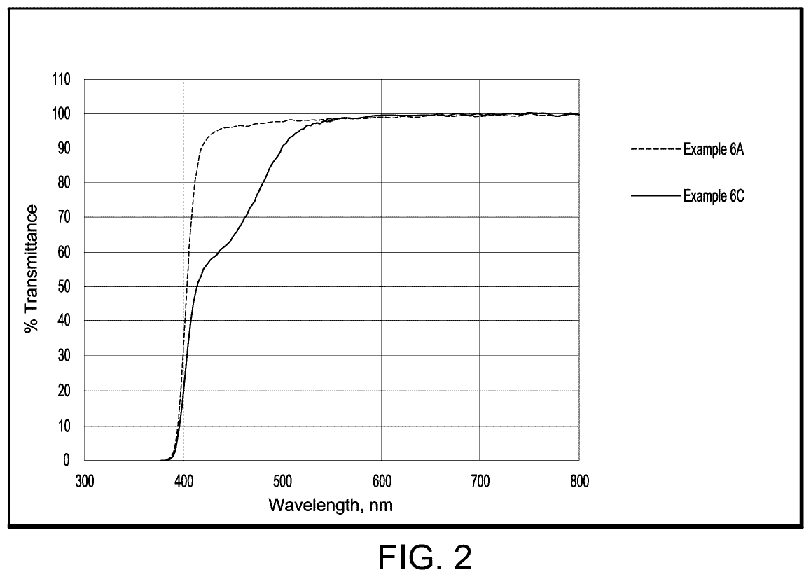

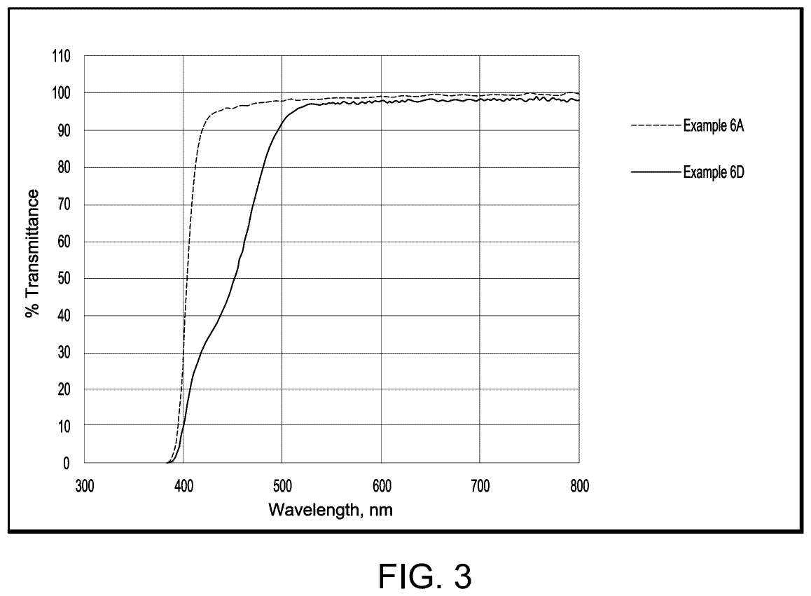

The components in Table 1 were vortex mixed in a 40 ml glass vial, purged with nitrogen, and then syringe filtered using a 1.0 micron and a 0.2 micron Acrodisc Teflon filter in series. Formulations were poured into polypropylene mold halves to prepare +34 diopter lenses or 0.5 mm thick discs. Samples were photo-cured for 2 to 6 hours using super actinic fluorescent bulbs (˜420 nm lambda max) with irradiance of ˜10-11 mW/cm². Test articles were extracted in acetone for 20 hours, air dried for 6 to 20 hours, and then vacuum dried at 80° C. for a minimum of 16 hours. The UV/Vis transmission spectra for the lenses obtained for Examples 6A-6D can be seen in FIGS. 1-3.

### Examples 7A-7D

The IOL formulations using a stock solution of Examples 6A and 6B made without initiator were mixed together with Irgacure 819 photo-initiator as shown below in Table 2 at amounts per wt. %, based on the total weight of the monomeric mixture.

TABLE 2

| Components | Ex. 7A | Ex. 7B | Ex. 7C | Ex. 7D |
|---|---|---|---|---|
| Ex. 6A | 74.74 | 50.10 | 24.83 | — |
| Ex. 6B | 24.86 | 49.56 | 74.88 | 99.54 |
| Irgacure 819 | 0.41 | 0.34 | 0.29 | 0.46 |

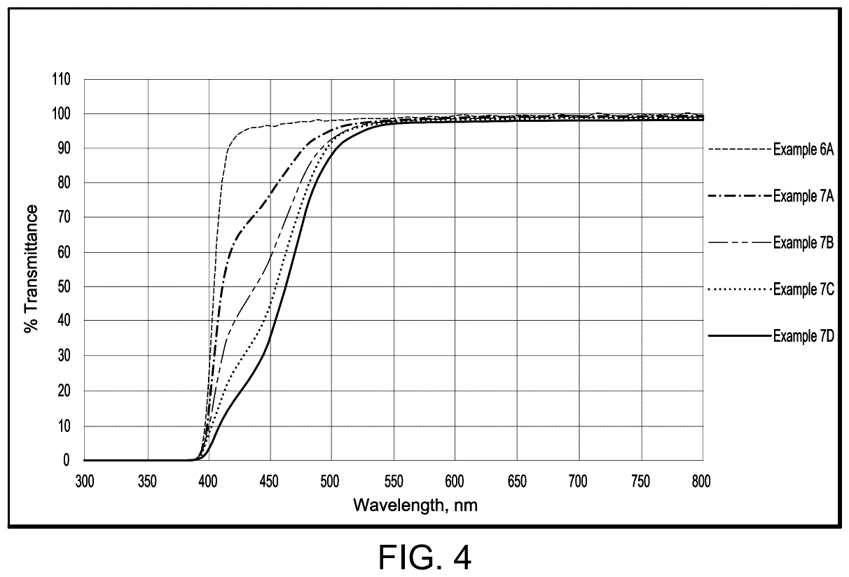

The components in Table 2 were vortex mixed in a 40 ml glass vial, purged with nitrogen, and then syringe filtered using a 1.0 micron and a 0.2 micron Acrodisc Teflon filter in series. Formulations were poured into polypropylene mold halves to prepare +34 diopter lenses or 0.5 mm thick discs. Samples were photo-cured for 2 to 6 hours using super actinic fluorescent bulbs (˜420 nm lambda max) with irradiance of ˜10-11 mW/cm². Test articles were extracted in acetone for 20 hours, air dried for 6 to 20 hours, and then vacuum dried at 80° C. for a minimum of 16 hours. The UV/Vis transmission spectra for the lenses obtained for Examples 7A-7D can be seen in FIG. 4.

The lenses made in Examples 7A-7D were tested for % extractables and equilibrium water content (EWC) at ambient temperature as shown in Table 3.

TABLE 3

| Example | % Extractables | EWC¹ (%) |
|---|---|---|
| Ex. 7A | 4.6 | 5.2 |
| Ex. 7B | 5.2 | 5.0 |
| Ex. 7C | 4.7 | 4.9 |
| Ex. 7D | 4.3 | 4.8 |

¹Extracted samples were equilibrated in deionized water for a minimum of 20 hours at ambient temperature.

In accordance with an aspect of the disclosure, a visible light absorbing azo compound has a structure of Formula I:

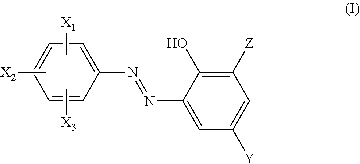

wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group.

In non-limiting illustrative embodiments, as may be combined with one or more of the preceding paragraphs, X₁ and X₂ are hydrogen, X₃ is a vinyl group, Y is a C₁ to C₆ alkyl group and Z is a hydrogen.

In non-limiting illustrative embodiments, as may be combined with one or more of the preceding paragraphs, X₁ and X₂ are hydrogen, X₃ is a C₁ to C₆ alkyl group, Y is a C₁ to C₆ alkyl group and Z is an allyl group.

In non-limiting illustrative embodiments, as may be combined with one or more of the preceding paragraphs, each of X₁, X₂ and X₃ are independently a C₁ to C₆ alkyl group, Y is a vinyl group and Z is a substituted or unsubstituted alkoxy group.

In non-limiting illustrative embodiments, as may be combined with one or more of the preceding paragraphs, X₁ and X₂ are hydrogen, X₃ is a C₁ to C₆ alkyl group, Y is a vinyl group or an allyl group and Z is a C₁ to C₆ alkoxy group.

In non-limiting illustrative embodiments, as may be combined with one or more of the preceding paragraphs, each of X₁, X₂ and X₃ are independently a C₁ to C₆ alkyl group, Y is a vinyl group or an allyl group and Z is a hydrogen.

In non-limiting illustrative embodiments, as may be combined with one or more of the preceding paragraphs, one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

In accordance with another aspect of the disclosure, an ophthalmic device is a polymerization product of a monomeric mixture comprising:
(a) a visible light absorbing azo compound having a structure of Formula I:

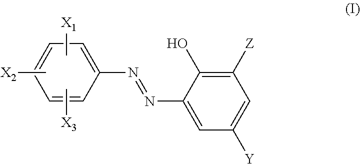

wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group, and
(b) one or more ophthalmic device-forming monomers.

In non-limiting illustrative embodiments, as may be combined with one or more of the preceding paragraphs, X₁ and X₂ are hydrogen, X₃ is a vinyl group, Y is a C₁ to C₆ alkyl group and Z is a hydrogen.

In non-limiting illustrative embodiments, as may be combined with one or more of the preceding paragraphs, in the visible light absorbing azo compound X₁ and X₂ are hydrogen, X₃ is a C₁ to C₆ alkyl group, Y is a C₁ to C₆ alkyl group and Z is an allyl group.

In non-limiting illustrative embodiments, as may be combined with one or more of the preceding paragraphs, in the visible light absorbing azo compound each of X₁, X₂ and X₃ are independently a C₁ to C₆ alkyl group, Y is a vinyl group and Z is a substituted or unsubstituted alkoxy group.

In non-limiting illustrative embodiments, as may be combined with one or more of the preceding paragraphs, in the visible light absorbing azo compound X₁ and X₂ are hydrogen, X₃ is a C₁ to C₆ alkyl group, Y is a vinyl group or an allyl group and Z is a C₁ to C₆ alkoxy group.

In non-limiting illustrative embodiments, as may be combined with one or more of the preceding paragraphs, in the visible light absorbing azo compound each of X₁, X₂ and X₃ are independently a C₁ to C₆ alkyl group, Y is a vinyl group or an allyl group and Z is a hydrogen.

In non-limiting illustrative embodiments, as may be combined with one or more of the preceding paragraphs, in the visible light absorbing azo compound one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

In non-limiting illustrative embodiments, as may be combined with one or more of the preceding paragraphs, the monomeric mixture comprises about 0.005 wt. % to about 0.5 wt. %, based on the total weight of the monomeric mixture, of the one or more visible light absorbers.

In non-limiting illustrative embodiments, as may be combined with one or more of the preceding paragraphs, the monomeric mixture comprises about 0.005 wt. % to about 0.2 wt. %, based on the total weight of the monomeric mixture, of the one or more visible light absorbers.

In non-limiting illustrative embodiments, as may be combined with one or more of the preceding paragraphs, the monomeric mixture comprises about 0.005 wt. % to about 0.1 wt. %, based on the total weight of the monomeric mixture, of the one or more visible light absorbers.

In non-limiting illustrative embodiments, as may be combined with one or more of the preceding paragraphs, the one or more ophthalmic device-forming monomers comprise styrene.

In non-limiting illustrative embodiments, as may be combined with one or more of the preceding paragraphs, the one or more ophthalmic device-forming monomers comprise one or more hydrophilic monomers.

In non-limiting illustrative embodiments, as may be combined with one or more of the preceding paragraphs, the one or more hydrophilic monomers are selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycol) functionalized with a polymerizable group and mixtures thereof.

In non-limiting illustrative embodiments, as may be combined with one or more of the preceding paragraphs, the one or more ophthalmic device-forming monomers comprise one or more polyethylene glycol methacrylate monomers.

In non-limiting illustrative embodiments, as may be combined with one or more of the preceding paragraphs, the one or more polyethylene glycol methacrylate monomers are represented by a structure of Formula II:

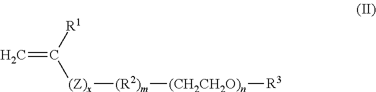

wherein R¹ is a hydrogen atom or a methyl radical; Z is a divalent group selected from the group —COO—, —CONH—, —CONCH₃—, —OCO—, —O—, —SO₂— —CO—O—CO— and —CO—CH₂—CO—; x is 0 or 1; R² is a linear, branched, cyclic, or aromatic, saturated or unsaturated, divalent carbon-based radical, of 1 to 30 carbon atoms; m is 0 or 1; n is an integer between 3 and 300 inclusive; and R³ is a hydrogen atom or a linear, branched, cyclic, or aromatic, saturated or unsaturated, carbon-based radical, of 1 to 30 carbon atoms.

In non-limiting illustrative embodiments, as may be combined with one or more of the preceding paragraphs, the one or more ophthalmic device-forming monomers comprise one or more heterocyclic monomers having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more monocyclic heterocyclic monomers having an ethylenically unsaturated reactive end group

In non-limiting illustrative embodiments, as may be combined with one or more of the preceding paragraphs, the one or more monocyclic heterocyclic monomers having an ethylenically unsaturated reactive end group comprise a C₃-C₈ heterocyclic monomer having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers represented by a structure of Formula III:

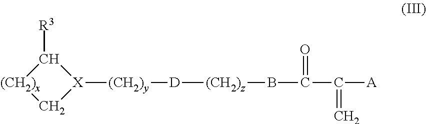

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃) 2; D is O, S, or a bond; A is H or CH₃; and z is 0 to 4, provided that if D is a bond then at least one of y and z is 0.

In non-limiting illustrative embodiments, as may be combined with one or more of the preceding paragraphs, x is an integer from 1 to 4; y is 0; R³ is an oxo group, X is N; D is O; z is an integer from 1 to 4; B is O; and A is H or CH₃.

In non-limiting illustrative embodiments, as may be combined with one or more of the preceding paragraphs, x is an integer from 1 to 3; y is 0; R³ is an oxo group, X is N; D is O; z is an integer from 2 to 3; B is O; and A is H or CH₃.

In non-limiting illustrative embodiments, as may be combined with one or more of the preceding paragraphs, x is an integer from 1 or 2; y is an integer from 1 to 4; R³ is an oxo group, X is N, D is a bond; z is 0; B is O; and A is H or CH₃.

In non-limiting illustrative embodiments, as may be combined with one or more of the preceding paragraphs, the one or more ophthalmic device-forming monomers comprise one or more aromatic monomers having an ethylenically unsaturated reactive end group.

In non-limiting illustrative embodiments, as may be combined with one or more of the preceding paragraphs, the one or more aromatic monomers having an ethylenically unsaturated reactive end group are a substituted or unsubstituted monoaromatic or polyaromatic radical containing from about 5 to about 25 carbon atoms.

In non-limiting illustrative embodiments, as may be combined with one or more of the preceding paragraphs, an aromatic group of the aromatic monomers having an ethylenically unsaturated reactive end group is a substituted or unsubstituted monoaromatic or polyaromatic radical containing from about 5 to about 25 carbon atoms

In non-limiting illustrative embodiments, as may be combined with one or more of the preceding paragraphs, an aromatic group of the aromatic monomers having an ethylenically unsaturated reactive end group is a substituted or unsubstituted monoaromatic radical containing from 6 to 12 carbon atoms directly bonded to an alkyl group.

In non-limiting illustrative embodiments, as may be combined with one or more of the preceding paragraphs, the one or more aromatic monomers having an ethylenically unsaturated reactive end group comprise one or more aromatic (meth)acrylic monomers.

In non-limiting illustrative embodiments, as may be combined with one or more of the preceding paragraphs, the (meth)acrylic group of the one or more aromatic (meth)acrylic monomers are (meth)acrylate-containing reactive end groups.

In non-limiting illustrative embodiments, as may be combined with one or more of the preceding paragraphs, the one or more aromatic (meth)acrylic monomer is selected from the group consisting of 2-phenylethyl acrylate, 2-phenylethyl methacrylate 2-phenoxyethyl acrylate, 2-phenoxyethyl methacrylate, 3-phenylpropyl acrylate, 3-phenylpropyl methacrylate and mixtures thereof.

In non-limiting illustrative embodiments, as may be combined with one or more of the preceding paragraphs, the monomeric mixture comprises about 50 wt. % to about 98 wt. %, based on the total weight of the monomeric mixture, of the one or more ophthalmic device-forming monomers.

In non-limiting illustrative embodiments, as may be combined with one or more of the preceding paragraphs, the monomeric mixture further comprises one or more crosslinking agents.

In non-limiting illustrative embodiments, as may be combined with one or more of the preceding paragraphs, the one or more crosslinking agents are selected from the group consisting of 1,4-butanediol diacrylate, 1,4-butanediol dimethacrylate, ethylene glycol dimethacrylate, diethylene glycol) diacrylate, diethylene glycol) dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate, poly(ethylene glycol) dimethacrylate, and poly(ethylene glycol) dimethacrylate.

In non-limiting illustrative embodiments, as may be combined with one or more of the preceding paragraphs, the monomeric mixture further comprises one or more ultraviolet light absorbing compounds.

In non-limiting illustrative embodiments, as may be combined with one or more of the preceding paragraphs, the one or more ultraviolet light absorbing compounds comprise benzotriazole ultraviolet light absorbing compounds.

In non-limiting illustrative embodiments, as may be combined with one or more of the preceding paragraphs, the ophthalmic device absorbs light between about 380 and about 500 nanometers (nm) wavelength.

In non-limiting illustrative embodiments, as may be combined with one or more of the preceding paragraphs, the light is blue light.

In accordance with yet another aspect of the disclosure, a method for making an ophthalmic device, comprises:
(a) providing a monomeric mixture comprising:
(i) a visible light absorbing azo compound having a structure of Formula I:

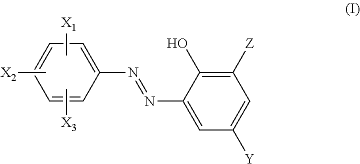

wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group, and
(ii) one or more ophthalmic device-forming monomers; and
(b) subjecting the monomeric mixture to polymerization conditions to provide an ophthalmic device.

In non-limiting illustrative embodiments, as may be combined with one or more of the preceding paragraphs, X₁ and X₂ are hydrogen, X₃ is a vinyl group, Y is a C₁ to C₆ alkyl group and Z is a hydrogen.

In non-limiting illustrative embodiments, as may be combined with one or more of the preceding paragraphs, in the visible light absorbing azo compound X₁ and X₂ are hydrogen, X₃ is a C₁ to C₆ alkyl group, Y is a C₁ to C₆ alkyl group and Z is an allyl group.

In non-limiting illustrative embodiments, as may be combined with one or more of the preceding paragraphs, in the visible light absorbing azo compound each of X₁, X₂ and X₃ are independently a C₁ to C₆ alkyl group, Y is a vinyl group and Z is a substituted or unsubstituted alkoxy group.

In non-limiting illustrative embodiments, as may be combined with one or more of the preceding paragraphs, in the visible light absorbing azo compound X₁ and X₂ are hydrogen, X₃ is a C₁ to C₆ alkyl group, Y is a vinyl group or an allyl group and Z is a C₁ to C₆ alkoxy group.

In non-limiting illustrative embodiments, as may be combined with one or more of the preceding paragraphs, in the visible light absorbing azo compound each of X₁, X₂ and X₃ are independently a C₁ to C₆ alkyl group, Y is a vinyl group or an allyl group and Z is a hydrogen.

In non-limiting illustrative embodiments, as may be combined with one or more of the preceding paragraphs, in the visible light absorbing azo compound one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

In non-limiting illustrative embodiments, as may be combined with one or more of the preceding paragraphs, the monomeric mixture comprises about 0.005 wt. % to about 0.5 wt. %, based on the total weight of the monomeric mixture, of the one or more visible light absorbers.

In non-limiting illustrative embodiments, as may be combined with one or more of the preceding paragraphs, the monomeric mixture comprises about 0.005 wt. % to about 0.2 wt. %, based on the total weight of the monomeric mixture, of the one or more visible light absorbers.

In non-limiting illustrative embodiments, as may be combined with one or more of the preceding paragraphs, the monomeric mixture comprises about 0.005 wt. % to about 0.1 wt. %, based on the total weight of the monomeric mixture, of the one or more visible light absorbers.

In non-limiting illustrative embodiments, as may be combined with one or more of the preceding paragraphs, the one or more ophthalmic device-forming monomers comprise styrene.

In non-limiting illustrative embodiments, as may be combined with one or more of the preceding paragraphs, the one or more ophthalmic device-forming monomers comprise one or more hydrophilic monomers.

In non-limiting illustrative embodiments, as may be combined with one or more of the preceding paragraphs, the one or more hydrophilic monomers are selected from the group consisting of an unsaturated carboxylic acid, an acrylamide, a vinyl lactam, a hydroxyl-containing-(meth)acrylate, a hydrophilic vinyl carbonate, a hydrophilic vinyl carbamate, a hydrophilic oxazolone, and a poly(alkene glycol) functionalized with a polymerizable group and mixtures thereof.

In non-limiting illustrative embodiments, as may be combined with one or more of the preceding paragraphs, the one or more ophthalmic device-forming monomers comprise one or more polyethylene glycol methacrylate monomers.

In non-limiting illustrative embodiments, as may be combined with one or more of the preceding paragraphs, the one or more polyethylene glycol methacrylate monomers are represented by a structure of Formula II:

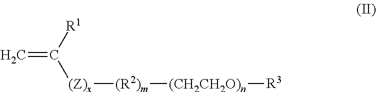

wherein R¹ is a hydrogen atom or a methyl radical; Z is a divalent group selected from the group —COO—, —CONH—, —CONCH₃—, —OCO—, —O—, —SO₂— —CO—O—CO— and —CO—CH₂—CO—; x is 0 or 1; R² is a linear, branched, cyclic, or aromatic, saturated or unsaturated, divalent carbon-based radical, of 1 to 30 carbon atoms; m is 0 or 1; n is an integer between 3 and 300 inclusive; and R³ is a hydrogen atom or a linear, branched, cyclic, or aromatic, saturated or unsaturated, carbon-based radical, of 1 to 30 carbon atoms.

In non-limiting illustrative embodiments, as may be combined with one or more of the preceding paragraphs, the one or more ophthalmic device-forming monomers comprise one or more heterocyclic monomers having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more monocyclic heterocyclic monomers having an ethylenically unsaturated reactive end group

In non-limiting illustrative embodiments, as may be combined with one or more of the preceding paragraphs, the one or more monocyclic heterocyclic monomers having an ethylenically unsaturated reactive end group comprise a C₃-C₈ heterocyclic monomer having an ethylenically unsaturated reactive end group.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers.

In non-limiting illustrative embodiments, as may be combined with one or more of the preceding paragraphs, the one or more heterocyclic monomers having an ethylenically unsaturated reactive end group comprise one or more heterocyclic (meth)acrylic monomers represented by a structure of Formula III:

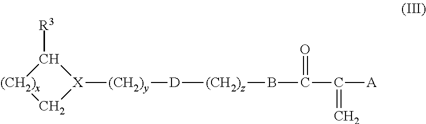

wherein x is an integer from 1 to 6, or from 1 to 5, or from 1 to 4 or from 1 to 2; y is an integer from 0 to 3 or from 1 to 3; R³ is hydrogen, hydroxy group, halogen group, carboxyl group, cyano group, nitro group, oxo (═O) group, thio (═S) group, a substituted or unsubstituted alkyl group, or a substituted or unsubstituted alkoxy group; X is a heteroatom such as O, N, S or P; B is O, NR, or S, where R is H, CH₃, CH₂CH₃, or CH(CH₃) 2; D is O, S, or a bond; A is H or CH₃; and z is 0 to 4, provided that if D is a bond then at least one of y and z is 0.

In non-limiting illustrative embodiments, as may be combined with one or more of the preceding paragraphs, x is an integer from 1 to 4; y is 0; R³ is an oxo group, X is N; D is O; z is an integer from 1 to 4; B is O; and A is H or CH₃.

In non-limiting illustrative embodiments, as may be combined with one or more of the preceding paragraphs, x is an integer from 1 to 3; y is 0; R³ is an oxo group, X is N; D is O; z is an integer from 2 to 3; B is O; and A is H or CH₃.

In non-limiting illustrative embodiments, as may be combined with one or more of the preceding paragraphs, x is an integer from 1 or 2; y is an integer from 1 to 4; R³ is an oxo group, X is N, D is a bond; z is 0; B is O; and A is H or CH₃.

In non-limiting illustrative embodiments, as may be combined with one or more of the preceding paragraphs, the one or more ophthalmic device-forming monomers comprise one or more aromatic monomers having an ethylenically unsaturated reactive end group.

In non-limiting illustrative embodiments, as may be combined with one or more of the preceding paragraphs, the one or more aromatic monomers having an ethylenically unsaturated reactive end group are a substituted or unsubstituted monoaromatic or polyaromatic radical containing from about 5 to about 25 carbon atoms.

In non-limiting illustrative embodiments, as may be combined with one or more of the preceding paragraphs, an aromatic group of the aromatic monomers having an ethylenically unsaturated reactive end group is a substituted or unsubstituted monoaromatic or polyaromatic radical containing from about 5 to about 25 carbon atoms

In non-limiting illustrative embodiments, as may be combined with one or more of the preceding paragraphs, an aromatic group of the aromatic monomers having an ethylenically unsaturated reactive end group is a substituted or unsubstituted monoaromatic radical containing from 6 to 12 carbon atoms directly bonded to an alkyl group.

In non-limiting illustrative embodiments, as may be combined with one or more of the preceding paragraphs, the one or more aromatic monomers having an ethylenically unsaturated reactive end group comprise one or more aromatic (meth)acrylic monomers.

In non-limiting illustrative embodiments, as may be combined with one or more of the preceding paragraphs, the one or more aromatic (meth)acrylic monomer is selected from the group consisting of 2-phenylethyl acrylate, 2-phenylethyl methacrylate 2-phenoxyethyl acrylate, 2-phenoxyethyl methacrylate, 3-phenylpropyl acrylate, 3-phenylpropyl methacrylate and mixtures thereof

In non-limiting illustrative embodiments, as may be combined with one or more of the preceding paragraphs, the monomeric mixture comprises about 50 wt. % to about 98 wt. %, based on the total weight of the monomeric mixture, of the one or more ophthalmic device-forming monomers.

In non-limiting illustrative embodiments, as may be combined with one or more of the preceding paragraphs, the monomeric mixture further comprises one or more crosslinking agents.

In non-limiting illustrative embodiments, as may be combined with one or more of the preceding paragraphs, the one or more crosslinking agents are selected from the group consisting of 1,4-butanediol diacrylate, 1,4-butanediol dimethacrylate, ethylene glycol dimethacrylate, diethylene glycol) diacrylate, diethylene glycol) dimethacrylate, triethylene glycol diacrylate, triethylene glycol dimethacrylate, tetraethylene glycol diacrylate, tetraethylene glycol dimethacrylate, 1,3-propanediol diacrylate, 1,3-propanediol dimethacrylate, 1,6-hexanediol diacrylate, 1,6-hexanediol dimethacrylate, poly(ethylene glycol) diacrylate, poly(ethylene glycol) dimethacrylate, and poly(ethylene glycol) dimethacrylate.

In non-limiting illustrative embodiments, as may be combined with one or more of the preceding paragraphs, the monomeric mixture further comprises one or more ultraviolet light absorbing compounds.

In non-limiting illustrative embodiments, as may be combined with one or more of the preceding paragraphs, the one or more ultraviolet light absorbing compounds comprise benzotriazole ultraviolet light absorbing compounds.

In non-limiting illustrative embodiments, as may be combined with one or more of the preceding paragraphs, the ophthalmic device absorbs light between about 380 and about 500 nanometers (nm) wavelength.

Various features disclosed herein are, for brevity, described in the context of a single embodiment, but may also be provided separately or in any suitable sub-combination. All combinations of the embodiments are specifically embraced by the illustrative embodiments disclosed herein just as if each and every combination was individually and explicitly disclosed. In addition, all sub-combinations listed in the embodiments describing such variables are also specifically embraced by the present compositions and are disclosed herein just as if each and every such sub-combination was individually and explicitly disclosed herein.

It will be understood that various modifications may be made to the embodiments disclosed herein. Therefore, the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. A visible light absorbing azo compound having a structure of Formula I: 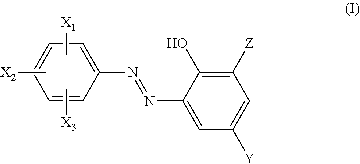
wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group.

2. The visible light absorbing azo compound according to claim 1, wherein X₁ and X₂ are hydrogen, X₃ is a vinyl group, Y is a C₁ to C₆ alkyl group and Z is a hydrogen.

3. The visible light absorbing azo compound according to claim 1, wherein X₁ and X₂ are hydrogen, X₃ is a C₁ to C₆ alkyl group, Y is a C₁ to C₆ alkyl group and Z is an allyl group.

4. The visible light absorbing azo compound according to claim 1, wherein each of X₁, X₂ and X₃ are independently a C₁ to C₆ alkyl group, Y is a vinyl group and Z is a substituted or unsubstituted alkoxy group.

5. The visible light absorbing azo compound according to claim 1, wherein X₁ and X₂ are hydrogen, X₃ is a C₁ to C₆ alkyl group, Y is a vinyl group or an allyl group and Z is a C₁ to C₆ alkoxy group.

6. The visible light absorbing azo compound according to claim 1, wherein each of X₁, X₂ and X₃ are independently a C₁ to C₆ alkyl group, Y is a vinyl group or an allyl group and Z is a hydrogen.

7. The visible light absorbing azo compound according to claim 1, wherein one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

8. An ophthalmic device which is a polymerization product of a monomeric mixture comprising:
(a) a visible light absorbing azo compound having a structure of Formula I: 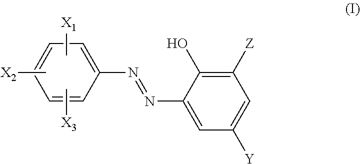
wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group; and
(b) one or more ophthalmic device-forming monomers.

9. The ophthalmic device according to claim 8, wherein in the visible light absorbing azo compound X₁ and X₂ are hydrogen, X₃ is a vinyl group, Y is a C₁ to C₆ alkyl group and Z is a hydrogen.

10. The ophthalmic device according to claim 8, wherein in the visible light absorbing azo compound X₁ and X₂ are hydrogen, X₃ is a C₁ to C₆ alkyl group, Y is a C₁ to C₆ alkyl group and Z is an allyl group.

11. The ophthalmic device according to claim 8, wherein in the visible light absorbing azo compound each of X₁, X₂ and X₃ are independently a C₁ to C₆ alkyl group, Y is a vinyl group and Z is a substituted or unsubstituted alkoxy group.

12. The ophthalmic device according to claim 8, wherein in the visible light absorbing azo compound X₁ and X₂ are hydrogen, X₃ is a C₁ to C₆ alkyl group, Y is a vinyl group or an allyl group and Z is a C₁ to C₆ alkoxy group.

13. The ophthalmic device according to claim 8, wherein in the visible light absorbing azo compound each of X₁, X₂ and X₃ are independently a C₁ to C₆ alkyl group, Y is a vinyl group or an allyl group and Z is a hydrogen.

14. The ophthalmic device according to claim 8, wherein in the visible light absorbing azo compound one of X₁, X₂ and X₃ is a vinyl group or an allyl group, and the other ones of X₁, X₂ and X₃ are hydrogen, Y is a vinyl group or an allyl group and Z is a hydrogen, a substituted or unsubstituted alkyl group, or a substituted or unsubstituted alkoxy group.

15. The ophthalmic device according to claim 8, wherein the monomeric mixture comprises about 0.005 wt. % to about 0.5 wt. %, based on the total weight of the monomeric mixture, of the visible light absorber.

16. The ophthalmic device according to claim 8, wherein the one or more ophthalmic device-forming monomers comprise one or more of styrene, a hydrophilic monomer, a polyethylene glycol methacrylate monomer, and a heterocyclic monomer having an ethylenically unsaturated reactive end group.

17. The ophthalmic device according to claim 8, wherein the ophthalmic device absorbs light between about 380 and about 500 nanometers (nm) wavelength.

18. The ophthalmic device according to claim 17, wherein the light is blue light.

19. The ophthalmic device according to claim 8, which is one of an intraocular lens, intraocular implant and a contact lens.

20. A method for making an ophthalmic device, comprising:
(a) providing a monomeric mixture comprising:
(i) a visible light absorbing azo compound having a structure of Formula I: 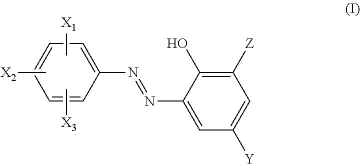
wherein X₁, X₂ and X₃ are independently hydrogen, a substituted or unsubstituted alkyl group, a substituted or unsubstituted alkoxy group, a vinyl group or an allyl group; Y is hydrogen, a substituted or unsubstituted alkyl group, or a vinyl group or an allyl group; and Z is hydrogen, a substituted or unsubstituted alkyl group, a substituted or unsubstituted alkoxy group, a vinyl group or an allyl group, wherein at least one of X₁, X₂, X₃, Y and Z is a vinyl group or an allyl group, and
(ii) one or more ophthalmic device-forming monomers; and
(b) subjecting the monomeric mixture to polymerization conditions to provide an ophthalmic device.
